(19) United States
(12) Patent Application Publication
(10) Pub. No.: US 2010/0191098 A1
(43) Pub. Date: Jul. 29, 2010

(54) METHOD TO TRACK A CONTRAST AGENT IN A MAGNETIC RESONANCE TOMOGRAPHY EXAMINATION

(76) Inventors: Susanne Ladd, Essen (DE); Michael Zenge, Nuernberg (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/692,079

(22) Filed: Jan. 22, 2010

(30) Foreign Application Priority Data
Jan. 23, 2009 (DE) 102009005903.2

(51) Int. Cl. A61B 5/055

(52) U.S. Cl. 600/420

(57) ABSTRACT

A method tracks contrast agent in a magnetic resonance tomography examination with examination table moving continuously in the Z-direction. In the method, a first magnetic resonance signal in a first magnetic resonance measurement without contrast agent. The first MR signal is acquired along a middle k-space line that runs essentially in the Z-direction. Values of k-space along the middle k-space line of the first MR signal are transformed by means of a Fourier transformation in the Z-direction in order to obtain a first profile of the signal intensity in the Z-direction. After a contrast agent injection, a second MR signal is acquired in a second magnetic resonance measurement. The second MR signal is acquired along the middle k-space line. Values of k-space along the middle k-space line of the second MR signal are transformed by a Fourier transformation only in the Z-direction in order to obtain a second profile of the signal intensity with contrast agent in the Z-direction. A difference profile is determined from the first profile and the second profile. A signal jump in the difference profile is used to determine a propagation edge of the contrast agent.

### BACKGROUND OF THE INVENTION

1. Field of the Invention

The present invention concerns a method to track a contrast agent in a magnetic resonance tomography examination and a corresponding magnetic resonance system. The method in particular concerns a tracking of the contrast agent in an examination with an examination table moving continuously in the Z-direction.

2. Description of the Prior Art

Particularly in recent years, contrast-enhanced magnetic resonance angiograms (Contrast Enhanced Magnetic Resonance Angiography, CE-MRA) have been accepted as clinical routine examinations. Fast gradient systems and an automatic table movement in combination with what is known as Total Imaging Matrix (Tim) technology support contrast agent tracking with high image quality, in particular in the region of renal arteries down to the veins in the feet. The Tim technology enables the three-dimensional, parallel data acquisition over large body regions or even the entire body in high quality, detail depth and anatomical coverage. This new data acquisition and reconstruction with a continuous table movement (TimCT) expands the possibilities of a peripheral magnetic resonance angiogram. The method enables the acquisition of seamless, large, observational spatial data with a significantly simplified workflow.

The temporal control of a contrast agent injection plays a decisive role in achieving a high artery signal in the arteries while avoiding venous signal overlays.

The contrast agent is typically injected in the form of a contrast agent bolus. After the contrast agent injection, the close temporal proximity of full arterial and venous phases requires that data acquisition must be implemented with greater temporal precision in order to prevent venous interferences.

In clinical practice, in many cases a test bolus measurement is therefore conducted before the actual bolus tracking measurement, which enables the arterial and venous time lapse to be predicted. This method is very reliable but requires the injection of an additional dose of a contrast agent, which reduces the allowed dose for the actual examination.

A manual fluoroscopic control reduces the contrast agent dose but requires a continuous monitoring and a precise intervention by the operator. Furthermore, this technique does not allow any suitable breath hold instructions.

Alternative, semi-automatic control methods are limited by the precise arrangement of a monitoring window over the vessels to be examined by the operator and are generally susceptible to movements. In particular in CE-MRA examinations with continuously moving examination table, conventional control methods are therefore insufficient since these methods do not reflect the significant variability of the blood speed along the peripheral vascular tree. A feedback of the leading propagation edge of the contrast agent bolus in the course of imaging in real time is therefore desirable in order to adapt the imaging parameters and the table speed to the current conditions.

### SUMMARY OF THE INVENTION

It is therefore the object of the present invention to provide a method to track a contrast agent in a magnetic resonance tomography examination which enables a fast tracking of a propagation edge of the contrast agent.

According to the present invention, a method is provided for tracking a contrast agent in a magnetic resonance tomography examination with an examination table moving continuously in the Z-direction. In the method, a first magnetic resonance signal is detected in a first magnetic resonance measurement without contrast agent. The first magnetic resonance signal is acquired along a middle k-space line that runs essentially in the Z-direction. Values of k-space along the middle k-space line of the first magnetic resonance signal are transformed with the aid of a Fourier transformation in the Z-direction and yield a first profile of the signal intensity in the Z-direction. After a contrast agent injection, a second magnetic resonance signal is detected in a second magnetic resonance measurement. The second magnetic resonance signal is likewise acquired along the middle k-space line. Values of k-space along the middle k-space line of the second magnetic resonance signal are transformed with the aid of a Fourier transformation only in the Z-direction and yield a second profile of the signal intensity in the Z-direction. According to the method, a difference profile is determined from the first profile and the second profile, in which difference profile the values of the first profile are subtracted from the values of the second profile at corresponding points in the Z-direction, for example. A propagation edge of the contrast agent is then determined from the difference profile.

The first magnetic resonance measurement is also designated as a native measurement and the second magnetic resonance measurement is designated as a bolus or contrast agent tracking measurement. The middle k-space line in the Z-direction pertains to values in k-space that are arranged along the Z-direction (i.e. in the limit frequency of the examination table) and essentially in the middle in the X-direction and Y-direction, i.e. in the middle of a plane perpendicular to the Z-direction in the examination region of a magnetic resonance system. Transformed values along the middle k-space line of the first MR measurement represent a background signal intensity of the examined subject along the Z-direction. Transformed values of the middle k-space line of the second MR measurement accordingly represent a profile of the background signal intensity plus the signal intensity due to the contrast agent. By determination of the difference profile, the background signal can be eliminated and regions with contrast agent and regions without contrast agent can thus be unambiguously differentiated. The propagation edge of the contrast agent can be determined in a simple manner from the transition between the region with contrast agent and the region without contrast agent. The transformation of the values of k-space along the middle k-space line can be implemented very quickly since the corresponding Fourier transformation is to be implemented only in the Z-direction.

In contrast to a conventional determination of an MR image in which the values of k-space are reconstructed in all two or three spatial directions with the aid of a Fourier transformation to reconstruct individual pixels of the MR image, according to the present invention the values of k-space of the second measurement are transformed only in the Z-direction and not in the other spatial direction(s) (X-direction and Y-direction). Since the middle k-space line represents the signal intensity along the Z-direction, a bolus tracking is possible solely using the information which is determined from the transformation of the values of k-space along the middle k-space linear of the second measurement in the Z-direction and the comparison with corresponding transformed values of the first measurement. Since both the measurement and the transformation as well as the determination of the propagation edge are implemented only in one dimension (in the Z-direction), a very fast tracking of the propagation edge is possible.

According to a further embodiment, additional second MR signals outside of the middle k-space line are additionally detected in the second MR measurement, and the values of k-space of the second measurement resulting from this are transformed by means of a Fourier transformation. An entire magnetic resonance image can thus be reconstructed from the second measurement. During the second measurement, the detection of the second MR signal along the middle k-space line can be implemented more frequently than the detection of the additional second MR signals which are acquired outside of the middle k-space line. The propagation edge of the contrast agent can thereby be re-determined continuously during the detection of the additional second MR signals, and the examination table can be positioned depending on the determined propagation edge of the contrast agent, for example. The acquisition quality of the reconstructed MR image in the region of the propagation edge of the contrast agent can thereby be determined particularly precisely. For example, for this the examination table can be moved depending on the determined propagation edge of the contrast agent such that the propagation edge is located approximately in a middle of a detectable examination region in the Z-direction.

According to a further embodiment, additional first MR signals outside of the middle k-space line are additionally detected in the first MR measurement, and values of k-space of the first measurement are transformed by means of a Fourier transformation. In addition to the first profile, a first complete MR image is thus reconstructed. A difference image which shows a spatial propagation of the contrast agent in the blood vessels of the examined subject can be determined by calculating a difference between the first MR image and a second MR image from the second measurement.

Furthermore, according to the present invention a magnetic resonance system is provided to track a contrast agent given an examination table moving continuously in the Z-direction. The magnetic resonance system has a control unit that operates a scanner and receives signals acquired by the scanner, and an evaluation device to evaluate the signals and generate an MR image. The magnetic resonance system is designed such that it detects a first MR signal without contrast agent in a first MR measurement. The first MR signal is acquired along a middle k-space line which runs essentially in the Z-direction. Values of k-space along the middle k-space line of the first MR signal are transformed by the magnetic resonance system with the aid of a Fourier transformation in the Z-direction. A profile of the signal intensity in the Z-direction results from this. After a contrast agent injection, a second MR signal is detected by the magnetic resonance system in a second MR measurement. The second MR signal is likewise acquired along the middle k-space line. Values of k-space along the middle k-space line of the second MR signal are then transformed by the magnetic resonance system with the aid of a Fourier transformation only in the Z-direction. A second profile of the signal intensity with contrast agent is thus determined in the Z-direction. The magnetic resonance system determines from the first profile and the second profile a difference profile in order to determine from this a propagation edge of the contrast agent. In further embodiments, the magnetic resonance system is designed such that it is suitable to implement the method described in the preceding.

The present invention also encompasses an electronically readable data medium—for example a CD or DVD—on which electronically readable control information (in particular software) is stored (encoded). When this control information is read from the data medium and stored in a control unit of the magnetic resonance system, all embodiments of the method described in the preceding according to the invention can be implemented with the magnetic resonance system.

### BRIEF DESCRIPTION OF THE DRAWINGS

FIG. 1 schematically illustrates a magnetic resonance system according to an embodiment of the present invention.

FIG. 2 schematically shows an angiogram which was acquired with the aid of a magnetic resonance tomograph; a data acquisition region of a magnetic resonance system; and a movement direction of an examination table of the magnetic resonance system.

FIG. 3 is a flowchart of a method to track a contrast agent in a magnetic resonance tomography examination.

FIG. 4 schematically shows signal intensity profiles which are determined in the method described in FIG. 3 for tracking a contrast agent.

### DESCRIPTION OF THE PREFERRED EMBODIMENTS

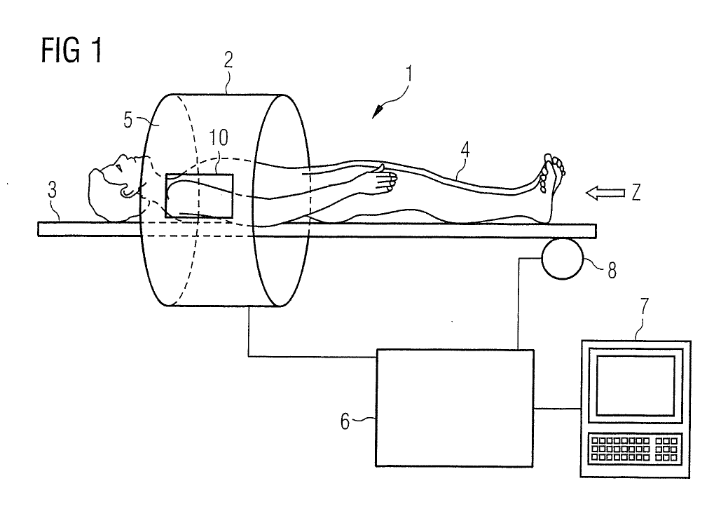

FIG. 1 shows a magnetic resonance system 1 that includes the actual scanner (data acquisition unit) 2, an examination table 3 for a patient 4 located in an opening 5 of the scanner 2, a control unit 6, an evaluation device 7 and a drive unit 8. The control unit 6 controls the scanner 2 and receives signals from the scanner 2 that are acquired by said scanner 2. Furthermore, the control unit 6 controls the drive unit 8 in order to move the examination table 3 together with the patient 4 along a direction Z through the opening 5 of the scanner 2. The evaluation device 7 evaluates the signals acquired by the scanner 2 to generate a magnetic resonance image (MR image). The evaluation device 7 is, for example, a computer system with a screen, a keyboard, a pointer input device (for example a mouse) and a data medium on which are stored electronically readable control information which is designed such that it implements the method described in the following for the tracking of a contrast agent in a magnetic resonance tomography examination upon use of the data medium in the evaluation device 7.

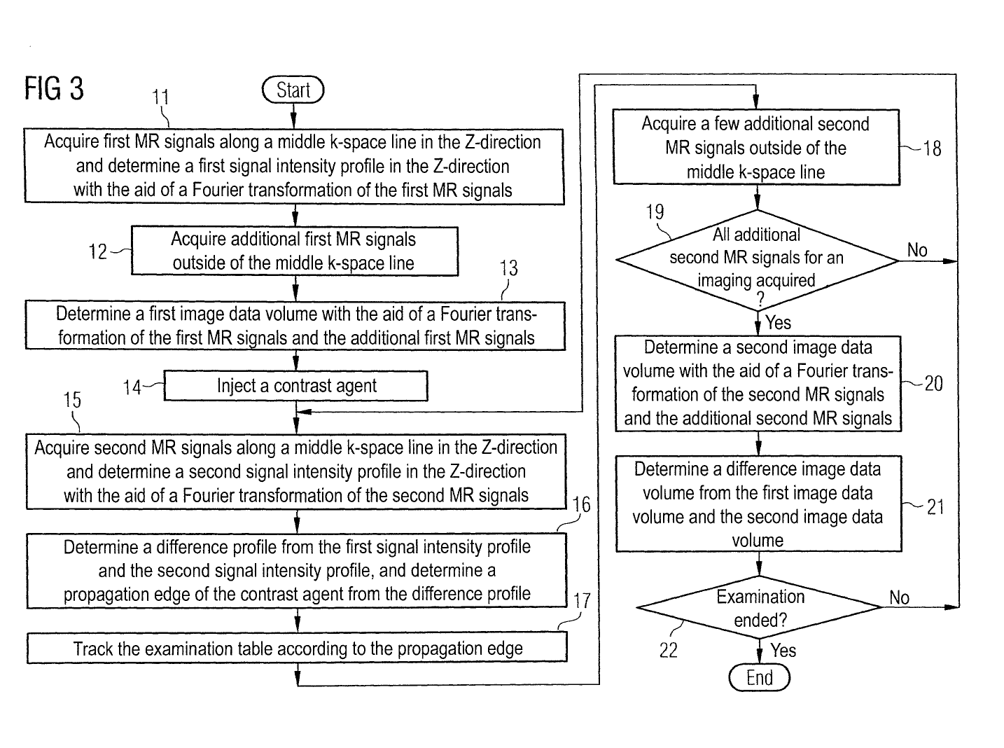

The method (which is described in the following with reference to FIG. 3) is in particular suitable to generate an angiogram using a contrast agent. The contrast agent is advantageously administered in the form of a contrast agent bolus.

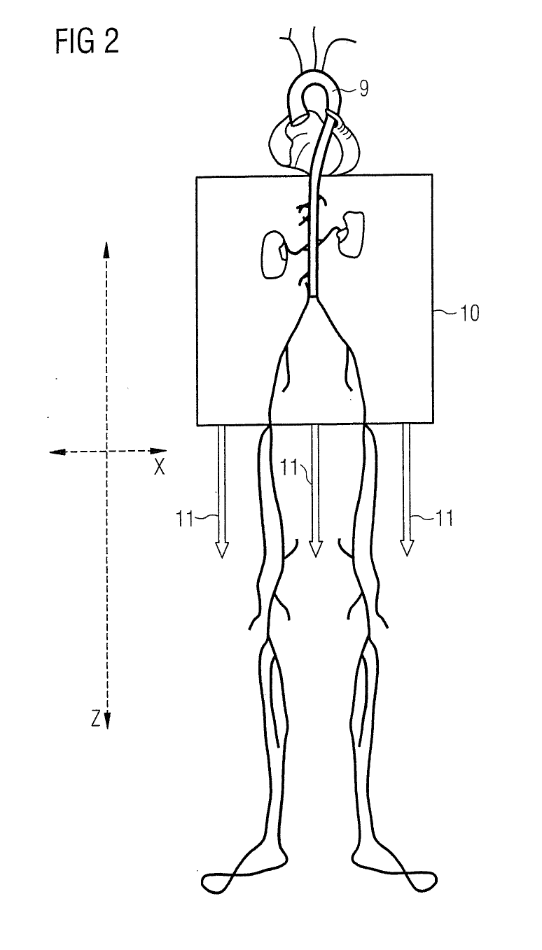

A coordinate system that is used in the following is initially defined with reference to FIG. 2. FIG. 2 shows an angiogram 9 which can be generated with the magnetic resonance system 1 shown in FIG. 1. The patient 4 is arranged on the examination table 3 with his body length in the Z-direction. The width of the patient (i.e. an extent of the patient along an axis that extends through both shoulders of the patient) runs in the X-direction. A Y-direction extends perpendicular to the X-direction and to the Z-direction. The magnetic resonance system 1 shown in FIG. 1 enables the examination of an examination region 10 within the opening 5 of the scanner 2 which extends both in the X/Y-direction and in the Z-direction. This examination region 10—which is also called a field of view (FOV)—is shown in FIG. 2 as a region 10 in the X/Z-plane. The examination region 10 can be displaced as indicated by the arrows 11 in the Z-direction by shifting the examination table 3.

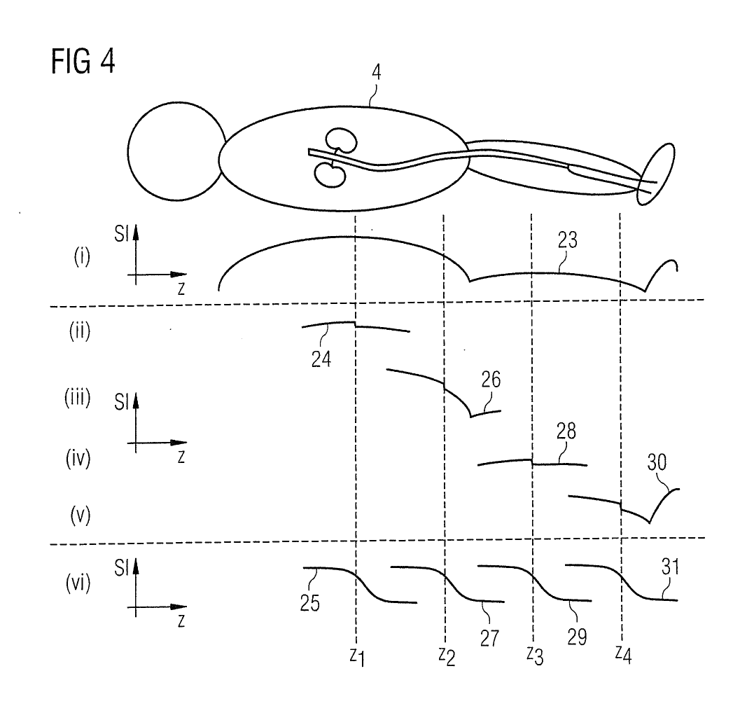

FIG. 3 shows a workflow diagram for a magnetic resonance angiography with a contrast agent tracking and an automatic examination table movement in combination with a total imaging matrix MR signal acquisition technology. First MR signals along a middle k-space line in the Z-direction are initially acquired in Step 11 in what is known as a native measurement (in which no contrast agent has been injected into the patient), and from these MR signals a first signal intensity profile is implemented in the Z-direction with the aid of a Fourier transformation of the first signals in the Z-direction. In order to obtain a first signal intensity profile over the entire length of the patient in the Z-direction, during acquisition of the first MR signals the patient 4 is moved continuously through the scanner 2. FIG. 4(i) shows an example of a signal intensity profile which is obtained from the acquired first MR signals with the aid of the Fourier transformation. Additional first MR signals outside of the middle k-space line are acquired in parallel with the acquisition of the first MR signals along the middle k-space line, and a first image data volume is generated with the aid of a Fourier transformation in the X-direction, Y-direction and Z-direction (Step 12 and 13).

In Step 14 a contrast agent is subsequently injected into the circulatory system of the patient 4, advantageously as a contrast agent bolus. Given an injection of the contrast agent into a bloodstream in the upper body of the patient, the predominant propagation direction of the contrast agent is initially in the direction of the feet of said patient. A propagation of the contrast agent is thus advantageously tracked in the direction of the arrows 11 of FIG. 2 in the Z-direction. For this second MR signals are detected in Step 15 along a middle k-space line in the Z-direction, and a second signal intensity profile in the Z-direction is determined in the Z-direction with a Fourier transformation of the second MR signals only in the Z-direction. A signal intensity profile in the Z-direction which is shown as a profile 24 in FIG. 4(ii) is thus determined for a current examination region, for example the examination region 10 shown in FIG. 2. At the point in time at which the signal intensity profile 24 was detected and determined, the contrast agent has propagated up to a position z₁ in the patient 4. Therefore a small jump in the signal intensity is to be detected in the signal intensity profile 24 at the point z₁. In Step 16, a difference profile 25 is determined from the first signal intensity profile 23 and the second signal intensity profile 24. The difference profile 25 is shown in FIG. 4(vi). Since the signal intensity curve in the Z-direction of the patient 4 has varied only by the signal of the contrast agent between the first measurement and the second measurement, using the difference profile it is very easy to determine where the propagation edge of the contrast agent is located. The examination table 3 can then be tracked—for example depending on the determined propagation edge of the contrast agent—so that the propagation edge of the contrast agent in the Z-direction is located centrally in the examination region 10 in order to achieve a best possible image quality of an MR image in the region of the propagation edge.

In Step 18, additional second MR signals outside of the middle k-space line can be detected which can subsequently be used for a reconstruction of an MR image. Since the contrast agent continuously propagates further during the acquisition of the additional second MR signals outside of the middle k-space line, the acquisition of these additional second MR signals outside of the middle k-space line is always interrupted again by an acquisition of MR signals along the middle k-space line in the Z-direction. The examination table 4 can thus be continuously tracked according to the propagation edge of the contrast agent with the aid of the MR signals along the middle k-space line and their transformation in the Z-direction. In Step 19 it is checked whether all signals outside of the middle k-space line have been detected for the reconstruction of a corresponding MR image. In the event that all MR signals have not yet been acquired, beginning with Step 15 MR signals along the middle k-space line to track the examination table 3 and signals outside of the middle k-space line are additionally detected in alternation. If all signals for a reconstruction of an MR image have been acquired, in Step 20 a second image data volume is determined with the aid of a Fourier transformation of the second MR signals. Finally, in Step 21 a difference image data volume is determined from the first image data volume without contrast agent and the second image data volume with contrast agent and is presented as an angiogram, for example on the evaluation device 7. The examination can subsequently be continued with Step 15 if a further tracking of the contrast agent and a generation of corresponding angiograms is desired (Step 22).

Signal intensity profiles 26, 28, 30 for additional positions of the examination table 3 and additional propagation states of the contrast agent are shown in FIG. 4(iii) through FIG. 4(v). The propagation edge of the contrast agent is located at z2 in FIG. 4(iii), such that the corresponding difference profile 27 at the point z2 exhibits a marked jump which characterizes the propagation edge of the contrast agent. FIG. 4(iv) shows the second signal intensity profile 28 at a still-later point in time at which the contrast agent is already located in the leg of the patient 4. The difference signal 28 accordingly shows a corresponding intensity jump at the position z3. Finally, in FIG. 4 the contrast agent has propagated just up to the foot of the patient 4, such that the second signal intensity profile 30 at the point z4 generates the signal intensity jump in the difference profile 31.

The acquisition of an MR signal along the middle k-space line in the Z-direction and a corresponding Fourier transformation only in the Z-direction can be implemented in a very short time span, for example within 100 ms, in contrast to which an acquisition of MR signals for an image reconstruction of the entire examination region 10 requires significantly more time (for example 10 s). A tracking of the contrast agent with the aid of the MR signals along the middle k-space line in the Z-direction is thus possible in real time. Moreover, the tracking of the propagation edge of the contrast agent requires only a very small amount of computing power since only a Fourier transformation in the Z-direction is required, and the propagation edge can be determined with the aid of a simple and one-dimensional examination of the difference profile. Furthermore, the method is independent of an illness of the patient since no prior knowledge whatsoever enters into the method to track the contrast agent.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for tracking a contrast agent in a magnetic resonance tomography examination comprising the steps of:
continuously moving an examination subject on an examination table through a magnetic resonance data acquisition unit in a movement direction corresponding to the Z-direction of a Cartesian coordinate system, and acquiring a first magnetic resonance signal from the examination subject, without administration of a contrast agent to the examination subject, in a first magnetic resonance data acquisition, along at least one middle line of k-space proceeding substantially in the Z-direction;
administering contrast agent to the examination subject and again continuously moving the examination subject through the data acquisition unit in said movement direction and acquiring a second magnetic resonance signal from the examination subject in a second magnetic resonance data acquisition, along said at least one middle line of k-space;
transforming values of k-space along said at least one middle line from the first magnetic resonance signal by Fourier transformation in the Z-direction, to obtain a first profile of the signal intensity in the Z-direction;
transforming values of k-space along said at least one middle line of said second MR signal by a Fourier transformation in the Z-direction, to obtain a second profile of the signal intensity in the Z-direction with contrast agent;
in a processor, determining a difference profile from said first profile and said second profile; and
in said processor, determining a propagation edge of said contrast agent in the examination subject from said difference profile.

2. A method as claimed in claim 1 comprising determining said propagation edge by Fourier transforming said k-space values along said at least one middle line for said second magnetic resonance signal only in said Z-direction to obtain said second profile.

3. A method as claimed in claim 1 comprising, in said second magnetic resonance data acquisition acquiring additional second magnetic resonance signals outside of said at least one middle like of k-space, and transforming said additional second magnetic resonance signals by Fourier transformation.

4. A method as claimed in claim 3 comprising acquiring said second magnetic resonance signal more frequently than acquiring said additional second magnetic resonance signals.

5. A method as claimed in claim 3 comprising acquiring additional first magnetic resonance signals outside of said of said at least one middle line of k-space in said first magnetic resonance data acquisition, and transforming all of the values in k-space from said first data acquisition with a Fourier transformation, and determining said difference profile from all of the transformed values of said first data acquisition and all of the transformed values of the second data acquisition.

6. A method as claimed in claim 1 comprising coordinating movement of said examination table in the movement direction dependent on said propagation edge of the contrast agent.

7. A method as claimed in claim 6 wherein said magnetic resonance data acquisition unit has a homogenous imaging volume, and coordinating said movement of said examination table to maintain said propagation edge of said contrast agent substantially in a middle of said examination volume.

8. A magnetic resonance system comprising:
a magnetic resonance data acquisition unit having a patient table and a control unit and a contrast agent injector;
continuously move an examination subject on said examination table through the magnetic resonance data acquisition unit in a movement direction corresponding to the Z-direction of a Cartesian coordinate system, and to acquire a first magnetic resonance signal from the examination subject, without administration of a contrast agent to the examination subject, in a first magnetic resonance data acquisition, along at least one middle line of k-space proceeding substantially in the Z-direction;
said control unit being configured to administer contrast agent to the examination subject and again continuously move the examination subject through the data acquisition unit in said movement direction and acquire a second magnetic resonance signal from the examination subject in a second magnetic resonance data acquisition, along said at least one middle line of k-space;
a processor configured to transform values of k-space along said at least one middle line from the first magnetic resonance signal by Fourier transformation in the Z-direction, to obtain a first profile of the signal intensity in the Z-direction, and to transform values of k-space along said at least one middle line of said second MR signal by a Fourier transformation in the Z-direction, to obtain a second profile of the signal intensity in the Z-direction with contrast agent;
said processor being configured to determine a difference profile from said first profile and said second profile, and to determine a propagation edge of said contrast agent in the examination subject from said difference profile.

9. An apparatus as claimed in claim 8 wherein said control unit is supplied with a signal from said processor representing said propagation edge, and is configured to coordinate movement of said examination table in the movement direction dependent on said propagation edge of the contrast agent.

10. An apparatus as claimed in claim 9 wherein said magnetic resonance data acquisition unit has a homogenous imaging volume, and wherein said control unit is configured to coordinate said movement of said examination table to maintain said propagation edge of said contrast agent substantially in a middle of said examination volume.

11. A computer-readable medium encoded with programming instructions for tracking a contrast agent in a magnetic resonance tomography examination in a magnetic resonance data acquisition unit operated by a computer system comprising a control unit, a processor an examination table, and a contrast agent injector, said programming instructions causing said computer system to:
operate said control unit to continuously move the examination subject on an examination table through a magnetic resonance data acquisition unit in a movement direction corresponding to the Z-direction of a Cartesian coordinate system, and to acquire a first magnetic resonance signal from the examination subject, without administration of a contrast agent to the examination subject, in a first magnetic resonance data acquisition, along at least one middle line of k-space proceeding substantially in the Z-direction;
operate said control unit to administer contrast agent from the contrast agent injector to the examination subject and to again continuously move the examination subject through the data acquisition unit in said movement direction, to acquire a second magnetic resonance signal from the examination subject in a second magnetic resonance data acquisition, along said at least one middle line of k-space;
operate the processor to transform values of k-space along said at least one middle line from the first magnetic resonance signal by Fourier transformation in the Z-direction, to obtain a first profile of the signal intensity in the Z-direction;
operate the processor to transform values of k-space along said at least one middle line of said second MR signal by a Fourier transformation in the Z-direction, to obtain a second profile of the signal intensity in the Z-direction with contrast agent;
operate the processor to determine a difference profile from said first profile and said second profile; and
operate the processor to determine a propagation edge of said contrast agent in the examination subject from said difference profile.
